(19) United States
(12) Patent Application Publication
(10) Pub. No.: US 2007/0299471 A1
(43) Pub. Date: Dec. 27, 2007

(54) Body-Worn Electrode Apparatus

(75) Inventors: Hisanori Takahashi, Ibarak-shi (JP); Hideo Sato, Ibaraki-shi (JP); Yoshio Funakoshi, Ibaraki-shi (JP); Masayuki Konno, Ibaraki-shi (JP); Hideki Okada, Nagoya-shi (JP); Tomoo Nakagawa, Nagoya-shi (JP)

Correspondence Address: HAMRE, SCHUMANN, MUELLER & LARSON, P.C., P.O. BOX 2902, MINNEAPOLIS, MN, 55402-0902, US

(73) Assignee: SUZUKEN CO., LTD., NAGOYA-SHI, AICHI (JP)

(21) Appl. No.: 10/577,959

(22) PCT Filed: Nov. 2, 2004
(86) PCT No.: PCT/JP04/16618

§ 371 Date: Mar. 8, 2007

(30) Foreign Application Priority Data
Nov. 4, 2003 (JP) 2003-374937

(51) Int. Cl. A61N 1/02

(52) U.S. Cl. 607/2

(57) ABSTRACT

A body-worn electrode apparatus 10 includes electrodes 1, 2 and 3 to be worn on the surface of a body, and a wiring 5 connected to the electrodes. At least a part of the wiring 5 includes a base material film 11 having a split induction part 13 capable of being split by hand, and a circuit 14 formed on a surface of the base material film into a shape so as to detour around the split induction part 13. The wiring 5 and the circuit 14 can be extended by spliting the split induction part 13.

### FIELD OF THE INVENTION

The present invention relates to a body-worn electrode apparatus for detecting an electric signal from a skin, and particularly to a body-worn electrode apparatus used when an electrocardiogram or the like is continuously measured over a specified time period.

### BACKGROUND ART

Conventionally, as one of electrode apparatuses for measuring electrocardiograms or the like, for example, there is known a type of electrode apparatus (so-called Holter electrode) which is worn on the chest over several hours to several days and continuously measures the electrocardiogram or the like during that period. In this kind of electrode apparatus, consideration is given to facilitating the movement of a user (patient or the like), and for example, the common type is such that electrodes are worn on three or four places, such as the center part of the chest, the upper part of the chest, and the flank.

Accordingly, in the electrode apparatus as stated above, it is necessary to accurately measure the electrocardiogram or the like by the three or four few electrodes, and it is desirable to make such a structure that the electrodes can be attached to suitable positions in accordance with the body shape of the user.

Besides, in order that the user wears it for a long period, it is desirable that even in the case where clothes are worn over the electrode apparatus which has been worn, there is little uncomfortable feeling.

Conventionally, in electrode apparatuses to be worn on the chest, as one in which a distance between electrodes can be adjusted, there is disclosed, for example, one as shown in FIG. 5, that is, an electrode apparatus 100 which includes an attachment base material 110 made of fiber, plural electrodes 101 formed by printing conductive material on the attachment base material 110, a circuit part 103, and an extendable part 102 in which slits 104 are alternately provided in the base material between the electrodes, and the circuit part 103 is formed into a zigzag form so as to weave through the slits 104 (see JP-UM-B-5-8967).

However, in the electrode apparatus as disclosed in JP-UM-B-5-8967, although the attachment position of the electrodes can be adjusted in accordance with the body shape of the user, as the electrode apparatus which has been described above and the user wears for a long period, problems as described below arise.

That is, in the electrode apparatus which the user wears for a long period, as described above, it is necessary that a small number of electrodes are spaced from each other to some degree and are worn. However, according to the length adjustment means using the slits as disclosed in JP-UM-B-5-8967, when the electrodes are spaced from each other, the “slack” is liable to occur in the circuit part, and the circuit part sways in the inside of the clothes and causes an uncomfortable feeling to the user, and further, the sway of the circuit part causes noise to be included in the electrocardiogram or the like.

Further, when the number of slits is decreased to prevent the “slack” from occurring, the “adjustment margin” of the electrode position is decreased, and it becomes difficult to attach the electrode to an accurate position in accordance with the body shape of the user, and consequently, there is a fear that an inaccurate electrocardiogram or the like is measured.

Then, the present invention provides a body-worn electrode apparatus in which the adjustment range of an electrode position is large, and even if the user wears it for a long period, the user hardly feels an uncomfortable feeling.

### DISCLOSURE OF THE INVENTION

A body-worn electrode apparatus of the invention includes an electrode to be worn on a surface of a body, and a wiring connected to the electrode. At least a part of the wiring includes a base material film having a split induction part, and a circuit formed on a surface of the base material film into a shape detouring around the split induction part. By splitting the split induction part, not only the wiring, but also the circuit can be extended.

According to the body-worn electrode apparatus having the structure described above, the user splits the base material film through the split induction part by a necessary amount in accordance with the body shape, so that the circuit formed to detour around the split induction part can be extended in the electrode direction by a necessary length. Since a portion that is not split is held on the base material film in a state where the circuit remains detouring around the split induction part, the unnecessary slack hardly occurs in the wiring, and it is possible to reduce the uncomfortable feeling in the case where the user wears it for a long period, and the noise due to the slack of the wiring.

Further, in the case where the circuit is formed on the base material film by printing, also when the portion that is not split is attached to the body, the wiring is not bulky, and also in this point, the uncomfortable feeling is hardly given to the user, and it becomes suitable for long time wearing.

Besides, since the split induction part is formed such that the base material film can be split by hand, there is an effect that even after the body-worn electrode apparatus is worn on the body, the user can easily finely adjust the length of the wiring in order to reduce the uncomfortable feeling.

Besides, in the body-worn electrode apparatus, the base material film and the circuit can be provided on a specified soft member. Similarly to the base material film, a split induction part can be provided also in this soft member. The split induction part of the soft member is formed in a state where it parallels the split induction part of base material film, in other words, in a state where the split induction part of the soft member and the split induction part of the base material film overlap with each other. By splitting the split induction part, not only the wiring, but also the circuit can be extended.

Here, the wiring can be constructed such that when the circuit is extended, the soft member can follow the extended circuit in a state where it has the same width as the base material film or is wider than the base material film within a range of 10 mm or less at one side.

Incidentally, the soft member here indicates a member softer than the base material film, that is, a member having a smaller elastic coefficient.

According to the body-worn electrode apparatus having the structure described above, the uncomfortable feeling of the user can be further reduced. That is, as the base material film, although a relatively hard raw material is often used to protect the printed circuit, the body-worn electrode apparatus can be worn on the skin through the soft member, and the stimulus to the skin can be reduced. Besides, even in the case where the wiring is twisted, the edge of the base material film hardly comes in contact with the skin, and the uncomfortable feeling of the user can be reduced by this.

Further, another (second) soft member can be laminated on an obverse side of the base material film, that is, on the electrode. Under this structure, the two soft members are disposed on the outermost surfaces of the wiring.

According to the structure described above, even in the case where the wiring is turned over, since the base material film does not come in direct contact with the skin, the uncomfortable feeling is further reduced.

Besides, at least part (segments of the circuit) of the circuit can be formed to be substantially parallel to each other at opposite positions across the split induction part.

According to the structure described above, when the body-worn electrode apparatus is manufactured, the base material film and the soft member can be efficiently used, and the manufacturing cost can be reduced. Further, since a portion not extended becomes compact, there is an effect that it hardly becomes a hindrance also at the time of attachment.

Besides, the circuit detouring around the split induction part may be preferably formed within a range of a horizontal to vertical ratio of 2 or less According to this structure as well, similarly to the above, it is possible to realize the reduction in cost by efficient use of the base material film and the like and the reduction in uncomfortable feeling by downsizing.

Further, an electrode base material film is provided on a surface of the electrode, and the whole width of the base material film constituting the part of the wiring can be set within a range of from 0.8 to 1.5 with respect to the whole width of the electrode base material film.

Incidentally, the whole widths of the base material film and the electrode base material film indicate the width orthogonal to the extension direction of the circuit.

According to the structure described above, when the electrode base material film provided on the surface of the electrode and the base material film constituting the part of the wiring are cut out from the same film material and are adopted, the film material is efficiently used, and the reduction in manufacturing cost can be realized.

As the split induction part, a perforated break line can be adopted.

When the split induction part is the perforated break line, the user can easily recognize its structure by a visual check or the like, and the usability is further improved. Besides, the manufacture of the body-worn electrode apparatus becomes easy.

The breaking strength of the perforated break line can be set to be from 0.2 to 5.0 per perforation.

According to the structure described above, the user can easily split the split induction part by hand, and it is possible to prevent the split induction part from being erroneously split.

### BRIEF DESCRIPTION OF THE DRAWINGS

FIG. 1 is a plan view showing an example of a body-worn electrode apparatus of the invention.

FIG. 2 is a schematic sectional view showing a structure of a circuit.

FIG. 3 is a view showing a state where the body-worn electrode apparatus of the embodiment is worn.

FIG. 4 is a sectional view showing a relation between a base material film and a soft member in an extended circuit.

FIG. 5 shows a conventional electrode apparatus constructed such that a distance between electrodes can be adjusted, in which (a) is a view showing a use state, and (b) is a view showing part of the electrode apparatus under magnification.

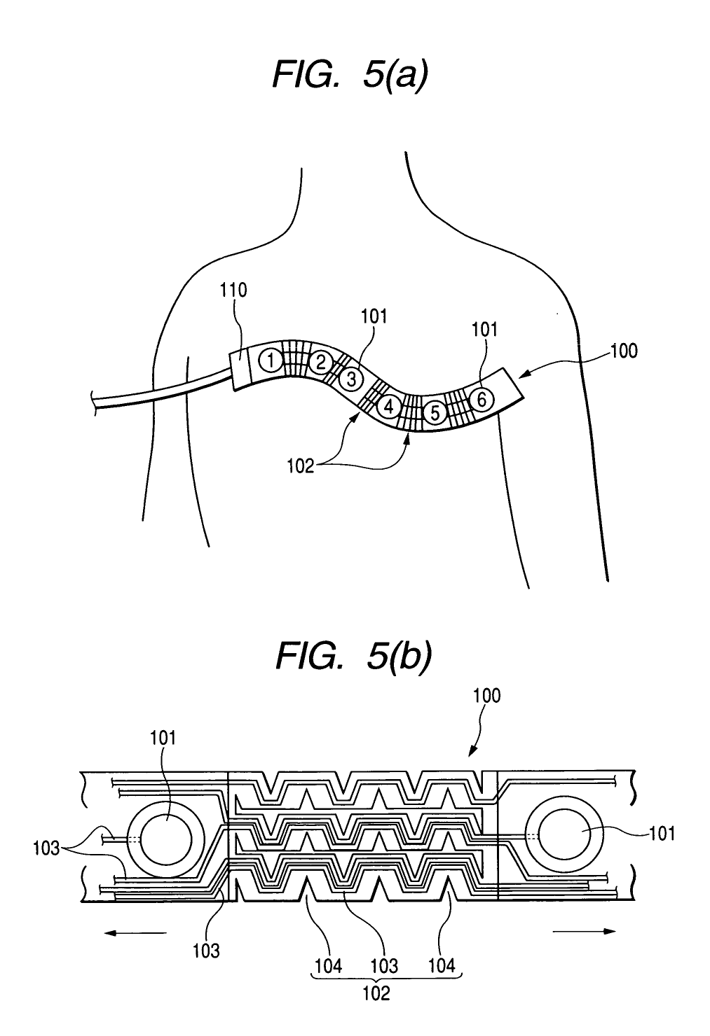

### BEST MODE FOR CARRYING OUT THE INVENTION

Hereinafter, embodiments of a body-worn electrode apparatus of the invention will be described.

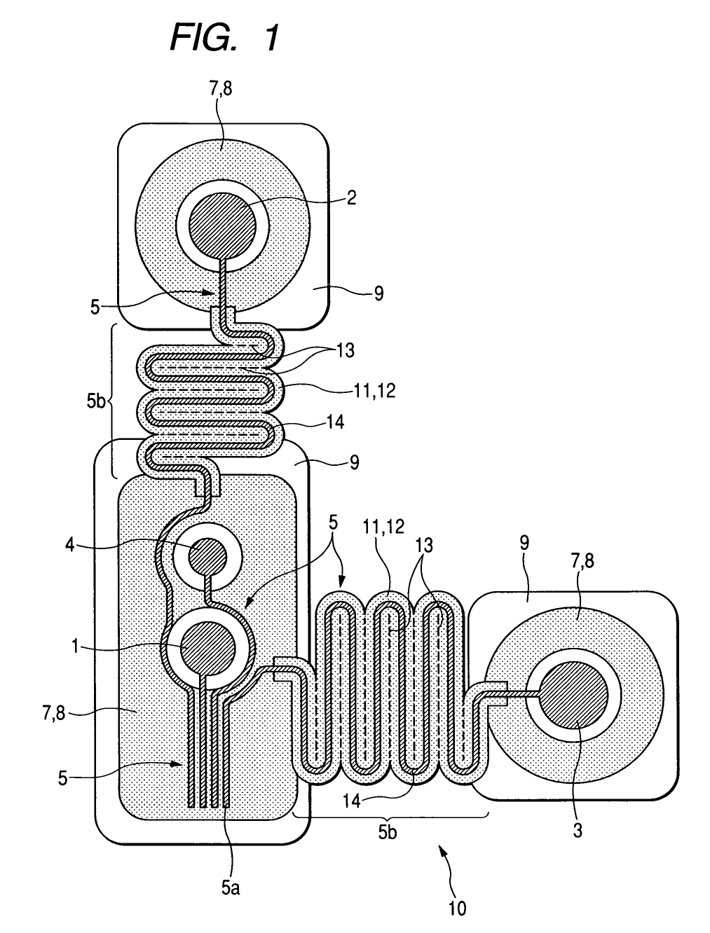

FIG. 1 is a plan view showing an example of a body-worn electrode apparatus 10 of the invention.

The body-worn electrode apparatus 10 of the embodiment includes a first electrode 1 attached to the center part of the chest, a second electrode 2 attached to the upper part of the chest, a third electrode 3 attached to the left flank, an earth electrode 4, and four wirings 5 connected to the respective electrodes. However, the wearing positions of the first electrode 1, the second electrode 2, and the third electrode 3 are not strictly limited. The respective electrodes are worn on the respective parts of the body according to the necessity of measurement.

Tip parts 5a of the wirings connected to the respective electrodes are concentrated in one place below the first electrode, and are constructed to be capable of being connected to, for example, a terminal (not shown) extending from a portable electrocardiogram recording apparatus.

In this embodiment, the first electrode 1, the earth electrode 4, and the tip part 5a of the wiring are fixed to the same electrode base material film 7 and nonwoven fabric 8, and are integrally constructed in a state where they are insulated from each other.

A length adjustment part 5b capable of adjusting the distance between the respective electrodes in accordance with the body shape of a user (patient) by extending the length of the wiring 5 is provided between the first electrode 1 and the second electrode 2, and between the first electrode 1 and the third electrode 3.

The length adjustment part 5b includes a base material film 11, an nonwoven fabric 12 constituting a soft member, and a circuit 14 formed of a conductive material printed on the base material film 11.

Various methods can be used as a print method of the circuit 14 onto the base material film 14, and a print method used for manufacture of various circuits, such as an integrated circuit and a flexible circuit, can be applied. Besides, the formation method of the circuit 14 is not limited to the print, and various methods used for circuit formation, such as an evaporation method and a plating method, can be used.

Perforated break lines 13 capable of being easily split by fingers are formed in the base material film 11 and the nonwoven fabric 12, and the circuit 14 is formed into a shape so as to detour around the perforated break lines 13. The perforated break lines 13 constitute a split induction part. The perforated break lines 13 can be formed at the same time in both the nonwoven fabric 12 and the base material film 11 by using a rotary cutter or the like. The break line of the nonwoven fabric 12 is formed in a state where it overlaps with the break line of the base material film 11.

The “split induction part” indicates a portion in the wiring 5, especially in the length adjustment part 5b, which can be torn mainly by hand and to which a specified process is applied. More specifically, in the length adjustment part 5b of the wiring 5 connected to the second electrode 2, groups of the three perforated break lines 13 parallel to each other in the horizontal direction, six in total, are alternately are formed in the base material film 11 and the nonwoven fabric 12. The circuit 14 is printed to meander horizontally so as to detour around the perforated break lines 13. Besides, the circuit 14 has a horizontal to vertical length ratio of substantially 1:1.5, and is formed such that the linear portions extending in the whole width direction (horizontal direction in FIG. 1) of the base material film are parallel to each other at the opposite positions across the break line 13.

On the other hand, also in the length adjustment part 5b of the wiring 5 connected to the third electrode 3, the seven, in total, perforated break lines 13 parallel to each other are alternately formed in the vertical direction in the base material film 11 and the nonwoven fabric 12, and the circuit 14 are printed to meander vertically so as to detour around the perforated break lines 13. Besides, the circuit 14 has a horizontal to vertical length ratio of substantially 1:1, and is formed such that the linear portions extending in the whole width direction (vertical direction in FIG. 1) of the base material film are parallel to each other at opposite positions across the break line 13.

In FIG. 1, the circuit 14, the length adjustment part 5b includes plural segments. The “segment” indicates a section from one bend place in the circuit 14 to a next bend. The two segments are disposed to be parallel to each other at both sides of the break line 13.

It is needless to say that the foregoing “parallel” does not mean strict parallel, and includes also the meaning of substantial parallel. Besides, at least one break line 13 is provided.

The length adjustment part 5b described above is constructed such that when the base material film 11 and the nonwoven fabric 12 are broken through the perforated break line 13, the base material film 11 and the nonwoven fabric 12 wider than the width of the extended circuit 14 by several mm follow the circuit 14.

It is preferable that the whole width of the base material film 11 constituting the length adjustment part 5b is within the range of 0.8 to 1.5 with respect to the whole width of the electrode base material film 7, and in this embodiment, it is about 0.83 at the second electrode side and about 1.1 at the third electrode side.

The perforated break line 13 formed in the base material film 11 is formed so that the breaking strength to split one perforation is preferably 0.2 to 5.0 (N/perforation).

Incidentally, the breaking strength here is a value measured as a maximum value at the time when upper and lower parts of one perforation are grasped in the breaking direction in Tensilon tensile test in conformance with JIS Z 0237, and are pulled at a pulling speed of 300 mm/min. When the breaking strength is set to be 0.2 N or higher per perforation, the electrode apparatus is smoothly manufactured, and when it is set to be 5.0 N or less, splitting by human hand is facilitated. However, it is not always necessary to set the breaking strength within this range.

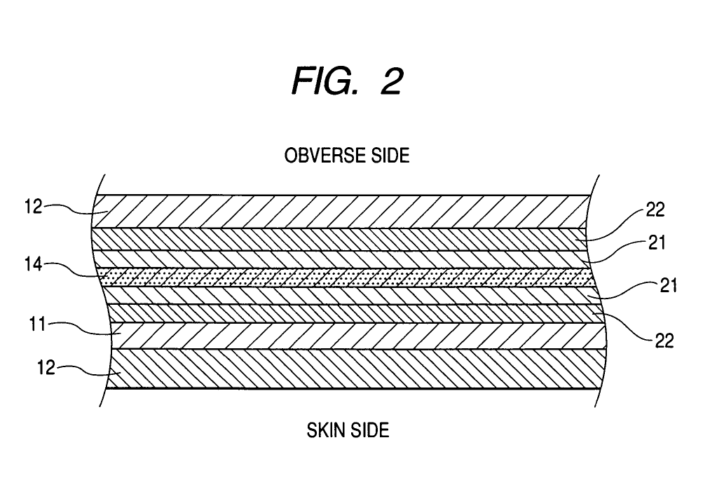

An electromagnetic wave shielding layer (conductive layer) to block high frequency noise and the like can be laminated on the circuit 14 formed by the print. Accordingly, for example, as shown in FIG. 2, the sectional structure of the wiring 5 is such that the circuit 14 is made the center, a pair of insulation layers 21 are laminated on and under the circuit 14, and further, a pair of electromagnetic wave shielding layers 22 (conductive layers) are laminated on and under the insulating layers 21, and the base material film 11 and the nonwoven fabric 12 are laminated to cover these.

Further, another nonwoven fabric (second soft member) 12 can be laminated on the obverse side of the upper base material film 11, and in this structure, the two nonwoven fabrics are disposed on the outermost surfaces of the wiring. According to the structure described above, even in the case where the wiring is turned over, since the base material film does not come in direct contact with the skin, the uncomfortable feeling is reduced.

Incidentally, as the need arises, an adhesive (pressure-sensitive adhesive) or an adhesive can be used for adhesion of the respective layers.

As the conductive material constituting the circuit 14, for example, what is obtained by mixing a conductive powder, such as a mixture of silver and silver chloride, with resin and solvent is preferably used.

As the insulating layer 21, although various resins, rubber, or ceramics can be used, from the viewpoint of superiority in insulation and flexibility, for example, polyimide resin or the like can be preferably used.

As the electromagnetic wave shielding layer 22, for example, what is constructed by mixing a conductive powder, such as carbon black, with resin and solvent and by printing can be preferably used.

Besides, as the base material film 11 used for the length adjustment part of the circuit and the electrode base material film 7 used for the electrode, various plastic films such as, for example, a PET film can be used. As the nonwoven fabrics 8 and 12 laminated around the electrode and on the reverse side of the circuit, for example, what is constructed of fiber of polyethylene, polypropylene, polyester or the like can be used.

On the other hand, each of the electrodes is constructed such that the electrode base material film 7 is the outermost layer (adverse side), and an electrode terminal is disposed at the reverse side (that is, the skin side) of the electrode base material film. As the soft member, the nonwoven fabric 8 punched into a doughnut shape is laminated around each of the electrode terminals and on the electrode base material film 7 through a double-sided tape. Besides, an adhesive layer for adhesion to the skin is laminated on the nonwoven fabric 8, and further, a release film 9 to cover the adhesive layer until it is used is laminated.

Further, as the electrode terminal, it is possible to preferably use an electrode terminal provided with an electrode plate constructed using a conductive material similar to the circuit, and a conductive skin adhesion member between the electrode plate and the skin (that is, the center of the doughnut-shaped nonwoven fabric 8). The electrode terminal and the skin are brought into contact with each other through the conductive skin adhesion member, so that the adhesiveness between the electrode terminal and the skin can be further raised. As the conductive skin adhesion member, for example, what is obtained by the gelation of water, glycerin, or electrolyte using water-soluble polymer can be preferably used.

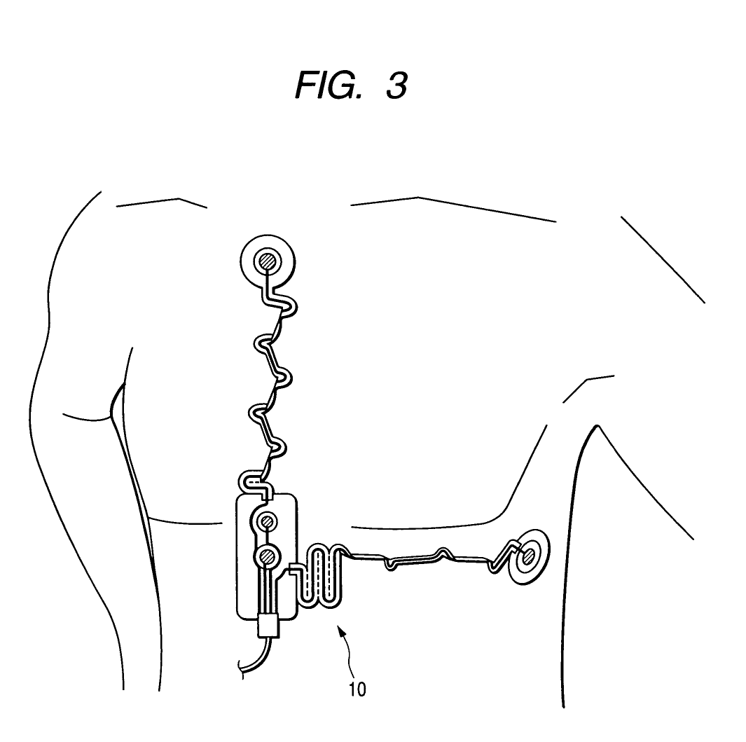

When the body-worn electrode apparatus 10 having the structure described above is used, first of all, after the first electrode 1 and the earth electrode 4 are attached to the center part (on the pit of the stomach) of the chest, the base material film 11 and the like are broken-through the perforated break line 13 so that the second electrode 2 is placed at an upper part of the chest, and the third electrode 3 is placed in the vicinity of the left flank, and the lengths of the respective circuits 14 are adjusted. As shown in FIG. 3, the respective electrodes are attached to the specified positions, so that the body-worn electrode apparatus 10 is worn on the chest of the user.

According to the body-worn electrode apparatus having the structure described above, since the circuit can be extended as the need arises, the electrodes can be attached to the accurate positions according to the height and girth of the user.

Besides, since the circuit can be extended by a necessary length, it is possible to prevent the circuit from slacking unnecessarily, and even in the case where the user wears it for a long period, there is little uncomfortable feeling.

Further, since the perforated break line is such that the base material film and the like can be easily split by hand, also in the case where the user feels uncomfortable during the use, the length of the circuit can be easily finely adjusted. Further, since the perforated break line can be easily formed, there is also an effect that the manufacturing cost of the body-worn electrode apparatus can be reduced.

Further, since the structure is made such that the whole width of the base material film 11 constituting the length adjustment part 5b falls within the range of 0.8 to 1.5 with respect to the whole width of the electrode base material film 7, when the base material film and the electrode base material film are manufactured by cutting the same film raw material, the film raw material can be effectively used, and the manufacturing cost of the body-worn electrode apparatus can be further reduced.

Incidentally, in the body-worn electrode apparatus of the embodiment, the example has been illustrated in which the three measuring electrodes arranged in the L shape when viewed in the flat, and the one earth electrode are provided, and the tip parts of the wirings are disposed substantially at the center part of the chest, however, the invention is not limited to the number of electrodes, the arrangement of those and the like as stated above.

Besides, in this embodiment, the example has been illustrated in which with respect to the circuit to be extended in the upward direction, the perforated break lines are formed horizontally and alternately, and the circuit meandering in the horizontal direction is provided so as to detour around the perforated break lines, and on the other hand, with respect to the circuit to be extended in the horizontal direction, the perforated break lines are formed vertically and alternately, and the circuit meandering in the vertical direction is provided so as to detour around the perforated break lines. However, the invention is not limited to the mode as stated above.

Thus, for example, also in a circuit to be extended in the vertical direction, perforated break lines are formed vertically and alternately, and a circuit meandering in the vertical direction may be provided so as to detour around these.

Besides, the shape of the circuit is not limited to a meandering one as in the above embodiment, and what is formed into an arbitrary shape, such as, for example, a zigzag shape, an arc shape, or a curl shape, can be adopted.

Further, in the above embodiment, although the perforated break lines are adopted as the split induction part, the invention is not limited to such a mode.

Thus, as the split induction part, for example, a structure may be made such that a linear body is attached to the base material film and the like along a part to be split, and the base material film can be split by peeling the linear body.

In the embodiment, although the nonwoven fabric is used as the soft member, a film raw material having a pleasant feel, such as a foamed material, an olefin film, a vinyl chloride film, or a polyurethane film can be used other than this.

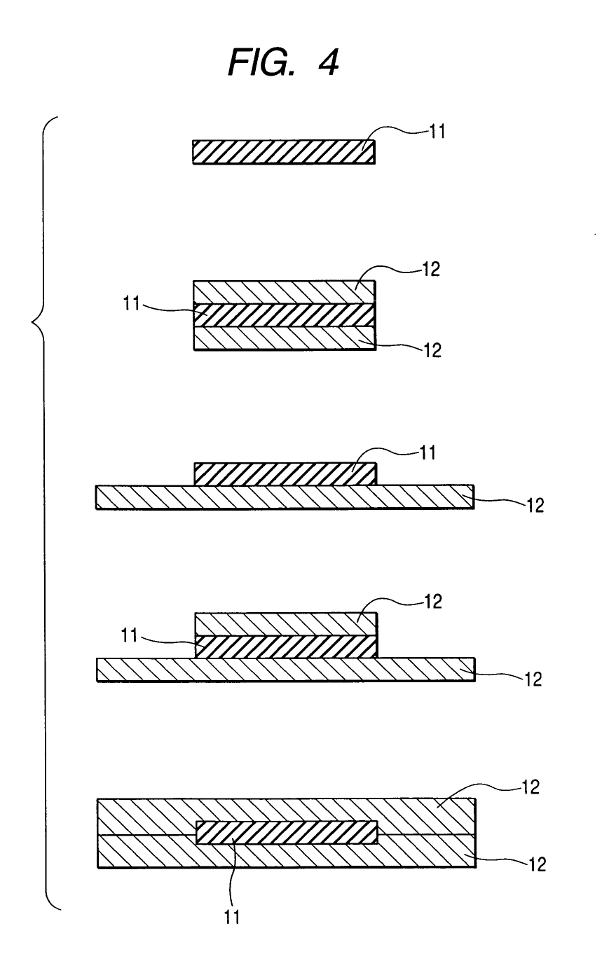

Besides, in the above embodiment, although the structure is made such that when the split induction part is split and the circuit is extended, the base material film and the nonwoven fabric (soft member) at the reverse side (that is, the skin side) have the same width, the invention is not limited to such a mode. That is, as shown in FIG. 4, a soft member of the same kind or different kind can be further laminated also on the obverse side of the base material film, and the these soft members can be made to have an arbitrary size.

In the case where the soft member is constructed to have the same width as the base material film or to be wider than the base material film, the contact between the base material film and the skin can be more certainly prevented in the extended circuit, and the uncomfortable feeling at the time of wearing can be further reduced. Incidentally, in this case, since it is necessary to make the width of the base material film thinner than the soft member, it is appropriate that the split induction part is formed in the soft member.

Besides, while the present invention has been described in detail and with reference to specific embodiments thereof, it will be apparent to one skilled in the art that various changes and modifications can be made therein without departing from the spirit and scope thereof.

The application is based on Japanese Patent Application (Patent Application No. 2003-374937) filed on Nov. 4, 2003, the contents thereof are incorporated herein by reference.

### INDUSTRIAL APPLICABILITY

According to the invention, the body-worn electrode apparatus is provided in which the wearing position of the electrode can be easily and accurately adjusted in accordance with the body shape of the user, and even if the user wears it for a long period, there is little uncomfortable feeling.

1. A body-worn electrode apparatus comprising:
an electrode to be worn on a surface of a body; and
a wiring connected to the electrode,
at least a part of the wiring including: a base material film having a split induction part; and a circuit formed on a surface of the base material film into a shape detouring around the split induction part.

2. The body-worn electrode apparatus according to claim 1,
wherein the circuit is printed on the base material film.

3. The body-worn electrode apparatus according to claim 1,
wherein at least a part of the wiring further includes a first soft member,
the base material film is disposed on the first soft member, and the first soft member includes a split induction part along the split induction part of the base material film.

4. The body-worn electrode apparatus according to claim 3,
wherein a second soft member is further laminated on the circuit, and the first soft member and the second soft member are disposed on outermost surfaces of the wiring.

5. The body-worn electrode apparatus according to claim 1,
wherein the circuit includes at least two segments disposed substantially in parallel to each other at opposite positions across the split induction part.

6. The body-worn electrode apparatus according to claim 1,
wherein the circuit detouring around the split induction part is formed within a range of a horizontal to vertical ratio of 2 or less.

7. The body-worn electrode apparatus according to claim 1,
wherein an electrode base material film is provided on a surface of the electrode, and a whole width of the base material film constituting a part of the wiring is set within a range of from 0.8 to 1.5 with respect to a whole width of the electrode base material film.

8. The body-worn electrode apparatus according to claim 1,
wherein the split induction part includes a perforated break line.

9. The body-worn electrode apparatus according to claim 8,
wherein breaking strength of the perforated break line is from 0.2 to 5.0 N per perforation.

10. The body-worn electrode apparatus according to claim 3,
wherein the soft member includes at least one selected from a nonwoven fabric, a foamed material, an olefin film, a vinyl chloride film, and a polyurethane film.
